(19) United States
(12) Patent Application Publication
(10) Pub. No.: US 2009/0312385 A1
(43) Pub. Date: Dec. 17, 2009

(54) CANNABINOID RECEPTOR MODULATORS FOR TREATING NON-IMMEDIATE TYPE ALLERGIC DISEASES

(75) Inventors: Shridhar Narayanan, Thane (west) (IN); Shobha Suryanarayana Rao, Navi Mumbai (IN)

Correspondence Address: Glenmark Pharmaceuticals S.A.;c/o Darby & Darby P.C., P.O Box 770, Church Street Station, New York, NY, 10008-0770, US

(73) Assignee: Glenmark Pharmaceuticals, S.A., La Chaux-de-Fonds (CH)

(21) Appl. No.: 12/510,652

(22) Filed: Jul. 28, 2009

Related U.S. Application Data
(63) Continuation-in-part of application No. PCT/IB2008/000164, filed on Jan. 25, 2008.
(60) Provisional application No. 60/910,873, filed on Apr. 10, 2007.

(30) Foreign Application Priority Data
Jan. 29, 2007 (IN) 157/MUM/2007

(51) Int. Cl. A61K 31/4162; A61P 37/08

(52) U.S. Cl. 514/403

(57) ABSTRACT

The present invention relates to the use of cannabinoid receptor modulators, particularly selective CB2 receptor agonists, for treating non-immediate type allergic diseases in mammals. The invention further relates to a pharmaceutical composition for non-immediate type allergic diseases.

### PRIORITY DOCUMENT(S)

This application is a continuation-in-part of International Application No. PCT/IB2008/000164, filed Jan. 25, 2008, which claims priority to Indian provisional patent application number 157/MUM/2007 filed on Jan. 29, 2007 and U.S. provisional application No. 60/910,873 filed on Apr. 10, 2007, all of which are hereby incorporated by reference.

### FIELD OF THE INVENTION

The present invention relates to the use of cannabinoid receptor modulators, more specifically Cannabinoid 2 receptor agonists for treating non-immediate type allergic diseases. The invention further relates to a pharmaceutical composition for treating non-immediate type allergic diseases.

### BACKGROUND OF THE INVENTION

The endogenous cannabinoid system comprises two main receptors, Cannabinoid 1 (“CB1”) and Cannabinoid 2 (“CB2”), and a number of ligands including anandamide and virodhamine which demonstrate the greatest activity at the cannabinoid receptor (Jonathan A W and Louis J A, Obes Man., 5-19, 2005). CB1 receptors are predominantly located in the brain and other neurons, while CB2 receptors are predominantly located in immune cells. Stimulation of these receptors is known to affect the central and peripheral action on lipid and glucose metabolism in adipose tissue and most notably, helps to regulate food intake, energy balance and nicotine dependence as well as regulate fear and anxiety.

There is evidence suggesting that CB1 agonists or antagonists, respectively, increase or decrease the motivation to work for palatable ingesta (Gallate J E and McGregor I S, Psychopharmacology, 142, 302-308, 1999 and Gallate J E, Saharov T, Mallet P E and McGregor I S, 1999, Eur. J. Pharmacol., 370, 233-240, 1990). Cannabinoids appear to directly stimulate eating by actions on appetitive processes, making food stimuli more salient and rapidly inducing eating even in satiated animals (Williams C M and Kirkham T C, Physiol. Behav., 76, 241-250, 2002).

Current data reveals that cannabinoids mediate suppression of inflammation in vitro and in vivo through stimulation of CB2 receptors (Ehrhart J, et. al. J. Neuroinflammation, 2, 29, 2005).

CB2 receptors are known to be expressed in the periphery. CB1 and CB2 receptor immunoreactivity has been demonstrated in cutaneous nerve fiber bundles, mast cells, macrophages, and epidermal keratinocytes in human skin sections (Rukwied, R. et al., J Dermatol Sci., 38(3):177-88 (2005)). There have been several reports of CB2 agonist being effective in a variety of models of inflammation, inflammatory pain and neuropathic pain (Hanus et al., Proc Natl Acad Sci USA., 96(25):14228-33 (1999)) and (Hohmann et al., Neuroscience, 119(3):747-57 (2003)).

K O Jonsson et al., have shown immuno-histochemical evidence of CB2 receptor in mouse epidermis (Basic Clin Pharmacol Toxicol., 98(2):124-34 (2006)). The same group also showed that a CB2 receptor agonist JWH133 reduced mast cell edema in response to compound 48/80 (K O Jonsson et al., Life Sci., 78(6):598-606 (2006)). Palmitoylethanolamide a putative CB2 receptor agonist was found to be effective in eosinophilic dermatitis (Noli C et al., Vet Dermatol., 12(1):29-39 (2001)).

PCT Application Publication No. WO 2006/129178, which is incorporated herein by reference in its entirety, discloses compounds of formula (I) described hereinafter as cannabinoid receptor modulators.

Some antiallergic effects of certain cannabinoids receptor modulators have been described in the literature. See, for example, US Publication No. 2004/0171613 and PCT Publication Nos. WO 02/010135, WO 03/061699, WO 2004/000807, WO 2006/046778, WO 96/18600, WO 96/25397, WO 99/57107, WO 00/10968, WO 01/4083, WO 01/19807, WO 01/28497, WO 02/42269, and WO 02/53543.

There is a need for safe and effective CB2 receptor agonists for treating skin disorders such as allergic dermatitis and atopic dermatitis. The present invention addresses this problem by providing safe and selective CB2 receptor agonists for treating non-immediate type allergic diseases such as atopic dermatitis, allergic asthma, and the like.

### SUMMARY OF THE INVENTION

The present invention relates to a use of cannabinoid receptor modulators, particularly those described in PCT Application Publication No. WO 2006/129178. The invention further relates to a pharmaceutical composition for non-immediate type allergic disease, and to a method for treating non-immediate type allergic disease.

Of the various cannabinoid receptor agonist compounds disclosed in the PCT Application Publication No. WO 2006/129178, those of particular interest, in the context of the present invention, include those which are selective CB2 receptor agonist. One such selective CB2 agonist is N5-(tert-butyl)-3-(2,4-difluorophenyl)-3,4-diazatricylo[5.2.1.0^{2,6}]deca-2(6),4-diene-5-carboxamide, including its pharmaceutically acceptable salts. Another such selective CB2 receptor agonist is (1R,7S)—N5-(tert-butyl)-3-(2,4-difluorophenyl)-3,4-diazatricylco[5.2.1.0^{2,6}]deca-2(6),4-diene-5-carboxamide (synonymously, the compound “GRC-CPD-A”) including its pharmaceutically acceptable salts.

An aspect of the present invention provides a method of treatment of non-immediate type allergic disease in a mammal by administering to the mammal a selective CB2 receptor agonist, such as N5-(tert-butyl)-3-(2,4-difluorophenyl)-3,4-diazatricylco[5.2.1.0^{2,6}]deca-2(6),4-diene-5-carboxamide, (1R,7S)—N-5-(tert-butyl)-3-(2,4-difluorophenyl)-3,4-diazatricylco[5.2.1.0^{2,6}]deca-2(6),4-diene-5-carboxamide or a pharmaceutically acceptable prodrug thereof, pharmaceutically acceptable N-oxide thereof, or pharmaceutically acceptable salt thereof.

Another aspect of the present invention provides a method of using a selective CB2 receptor agonist, such as N5-(tert-butyl)-3-(2,4-difluorophenyl)-3,4-diazatricylco[5.2.1.0^{2,6}]deca-2(6),4-diene-5-carboxamide, including its pharmaceutically acceptable salts. (1R,7S)—N5-(tert-butyl)-3-(2,4-difluorophenyl)-3,4-diazatricylco[5.2.1.0^{2,6}]deca-2(6),4-diene-5-carboxamide or pharmaceutically acceptable prodrug thereof, pharmaceutically acceptable N-oxide thereof, or pharmaceutically acceptable salt thereof for the treatment of non-immediate type allergic disease in a mammal, wherein the cannabinoid receptor agonist is administered to the mammal by the oral, parenteral or topical route.

A further aspect of the present invention provides a pharmaceutical formulation comprising a selective CB2 receptor agonist, such as N5-(tert-butyl)-3-(2,4-difluorophenyl)-3,4-diazatricylco[5.2.1.0^{2,6}]deca-2(6),4-diene-5-carboxamide, (1R,7S)—N5-(tert-butyl)-3-(2,4-difluorophenyl)-3,4-diazatricylco[5.2.1.0^{2,6}]deca-2(6),4-diene-5-carboxamide or pharmaceutically acceptable prodrug thereof, pharmaceutically acceptable N-oxide thereof, or pharmaceutically acceptable salt thereof optionally together with one or more pharmaceutically acceptable excipients, carriers, diluents or mixtures thereof, for treatment of non-immediate type allergic disease in mammal.

Yet another aspect of the present invention provides use of a selective CB2 receptor agonist, such as N5-(tert-butyl)-3-(2,4-difluorophenyl)-3,4-diazatricylco[5.2.1.0^{2,6}]deca-2(6),4-diene-5-carboxamide, (1R,7S)—N5-(tert-butyl)-3-(2,4-difluorophenyl)-3,4-diazatricylco[5.2.1.0^{2,6}]deca-2(6),4-diene-5-carboxamide or pharmaceutically acceptable prodrug thereof, pharmaceutically acceptable N-oxide thereof, or pharmaceutically acceptable salt thereof for preparing a pharmaceutical formulation for treatment of non-immediate type allergic disease in mammal.

A further aspect of the present invention relates to a process of preparing a pharmaceutical formulation comprising N5-(tert-butyl)-3-(2,4-difluorophenyl)-3,4-diazatricylco[5.2.1.0^{2,6}]deca-2(6),4-diene-5-carboxamide, (1R,7S)—N5-(tert-butyl)-3-(2,4-difluorophenyl)-3,4-diazatricylco[5.2.1.0^{2,6}]deca-2(6),4-diene-5-carboxamide or a pharmaceutically acceptable prodrug thereof, pharmaceutically acceptable N-oxide thereof, or pharmaceutically acceptable salt thereof for the treatment of non-immediate type allergic disease in a mammal.

According to another embodiment of the present invention, the non-immediate type allergic disease is selected from, for example, allergic asthma, allergic dermatitis, atopic dermatitis, allergic rhinitis, allergic conjunctivitis or a combination thereof. Particularly, the non-immediate type allergic disease is allergic asthma, allergic dermatitis, or atopic dermatitis, either alone or as combination in the subject.

In another embodiment of the present invention, the mammal is a human.

### BRIEF DESCRIPTION OF THE DRAWINGS

FIG. 1: Effect of GRC-CPD-A on percent ear swelling in TDI-induced dermatitis in BALB/c mice.

FIG. 2: Effect of GRC-CPD-A on histology scores in TDI-induced dermatitis in BALB/c mice.

FIG. 3: Effect of GRC-CPD-A on total number of eosinophils/12 hpf in TDI-induced dermatitis in BALB/c mice.

FIG. 4: Effect of GRC-CPD-A on eosinophils peroxidase levels in TDI-induced dermatitis in BALB/c mice.

### DETAILED DESCRIPTION OF THE INVENTION

In general, the present invention relates to the use of a compound of formula (I), and a pharmaceutical composition containing it for treating a non-immediate type allergic disease, and to a method of treating a non-immediate type allergic disease using a compound of formula (I).

The compound of formula (I) described herein is represented by:

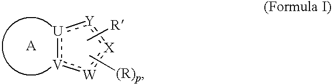

pharmaceutically acceptable prodrugs thereof, pharmaceutically acceptable N-oxides thereof, and pharmaceutically acceptable salts thereof,
wherein:

each of the dotted lines in formula (I) independently represents an optional bond;

U and V are independently C or N;

W, X and Y are independently C, N, O, S or —C(O)—;

each occurrence of R and R′ may be same or different and are independently alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, heterocyclylalkyl, cyano, hydroxyl, halogen, C(O)NHNH₂, C(═B)NR¹R², C(O)NHNHC(O)R¹, C(═O)NH₂, C(═S)NH₂, C(═B)NH(CR¹R²)ₙ.OH, C(═NR¹)R²(CH₂)ₚNR¹R², (CH₂)ₚCR¹R², CH═CR¹R², CF₂R¹, CHFR¹, (CH₂)ₚOR¹, C(═B)R¹, C(O)OR¹, C(O)N(OR¹)R², NR¹CONR¹R²S(O)ₘR¹, S(O)ₘNR¹R¹, NR¹COR², NR¹CSR², NR¹SO₂R², C(═NR¹)NR¹R², C(═NOR¹)R², C(═NNR¹)R² or C(OR¹)R²;

each occurrence of R¹ and R² may be same or different and are independently hydrogen, nitro, halo, cyano, —OR³, —SR³, oxo, thio, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic group, substituted or unsubstituted heterocyclylalkyl, —C(═B)—R³, —C(O)O—R³, —C(O)NR³R⁴, —S(O)ₘ—R⁴, —S(O)ₘ—NR³R⁴, —NR³R⁴, or a protecting group or R¹ and R², when bound to a common atom, may be joined together to form an optionally substituted 3 to 7 membered saturated or unsaturated cyclic ring, which may optionally include at least two heteroatoms selected from O, NR³ or S;

each occurrence of R³ and R⁴ may be same or different and are independently hydrogen, halogen, nitro, cyano, formyl, acetyl, oxo, thio, —C(O)—R^{a}, —C(O)O—R^{a}, —C(O)NR^{a}R^{b}, —S(O)ₘ—R^{a}, —S(O)ₘ—NR^{a}R^{b}, —NR^{a}R^{b}, —OR^{a}, —SR^{a}, a protecting group, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalklenylalkyl, substituted or unsubstituted aryl, substituted or unsubstituted arylalkyl, substituted or unsubstituted heteroaryl, substituted or unsubstituted heterocyclic group, substituted or unsubstituted heterocyclylalkyl, or substituted or unsubstituted heteroarylalkyl;

each occurrence of R^{a} and R^{b} may be same or different and are independently hydrogen, halogen, nitro, cyano, formyl, acetyl, oxo, thio, a protecting group, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclic group, substituted or unsubstituted heterocyclylalkyl, or substituted or unsubstituted heteroarylalkyl;

each occurrence of B is independently O, S or NR³;

p and m are independently 0, 1 or 2;

A is

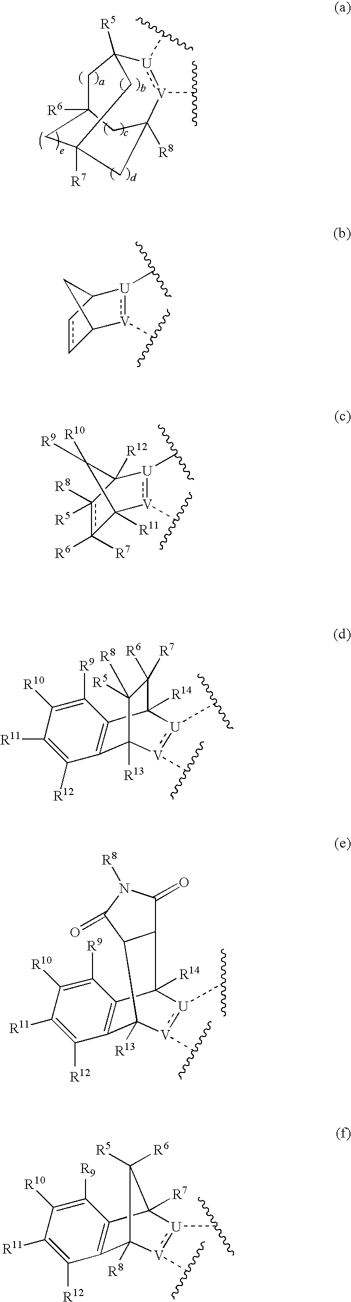

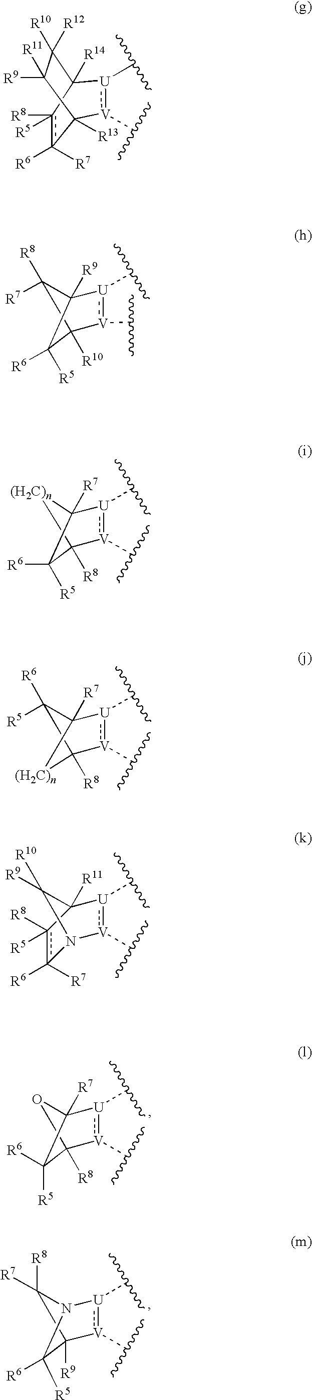

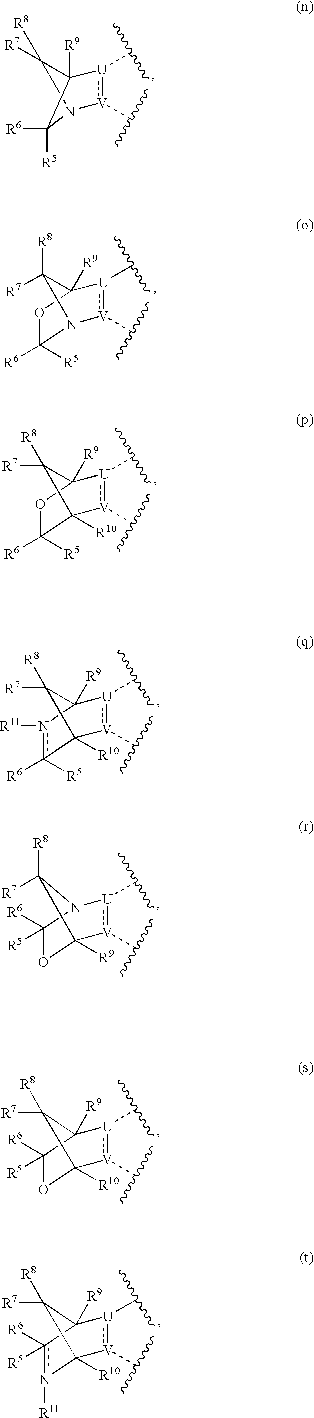

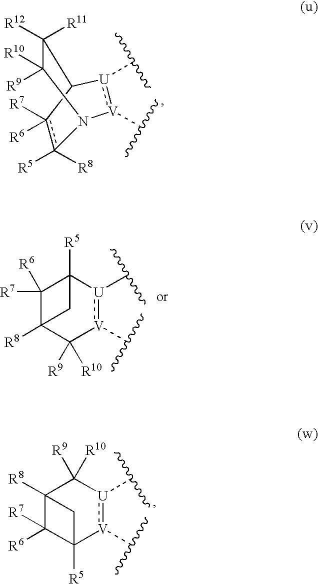

wherein:

each of the dotted lines in A independently represents an optional bond;

R⁵, R⁶, R⁷, R⁸, R⁹, R¹⁰, R¹¹, R¹², R¹³, and R¹⁴ are independently hydrogen, nitro, cyano, formyl, acetyl, halogen, —OR¹⁵, —SR¹⁵, oxo, thio, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic group, substituted or unsubstituted heterocyclylalkyl, —NR¹⁵R¹⁶, —C(═B)—R¹⁵, —C(O)O—R¹⁵, —C(O)NR¹⁵R¹⁶, —S(O)ₘ—R¹⁵, —S(O)ₘ—NR¹⁵R¹⁶, or a protecting group;

R⁵ and R⁶ may be joined together to form an optionally substituted 3 to 11 membered saturated or unsaturated mono or bicyclic ring, which may optionally include at least one heteroatom selected from O, NR³ or S;

R⁹ and R¹⁰ may be joined together to form an optionally substituted 3 to 11 membered saturated or unsaturated mono or bicyclic ring, which may optionally include at least one heteroatom selected from O, NR³ or S;

R⁵ and R⁹ may be joined together to form an optionally substituted 3 to 11 membered saturated or unsaturated mono or bicyclic ring, which may optionally include at least one heteroatom selected from O, NR³ or S;

R⁷ and R¹⁰ may be joined together to form an optionally substituted 3 to 11 membered saturated or unsaturated mono or bicyclic ring, which may optionally include at least one heteroatom selected from O, NR³ or S;

each occurrence of R¹⁵ and R¹⁶ may be same or different and are independently hydrogen, nitro, halo, cyano, —OR³, —SR³, oxo, thio, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic group, substituted or unsubstituted heterocyclylalkyl, —C(═B)—R³, —C(O)O—R³, —C(O)NR³R⁴, —S(O)ₘ—R³, S(O)ₘ—NR³R⁴, —NR³R⁴ or R¹⁵ and R¹⁶, when bound to a common atom, may be joined together to form an optionally substituted 3 to 7 membered saturated or unsaturated cyclic ring, which may optionally include at least two heteroatoms selected from O, NR³ or S wherein R³ and R⁴ are as defined as above;

n is 1, 2, 3, or 4; and

a, b, c, d and e are integers independently selected from 0 to 4.

The term “aryl” refers to aromatic radicals having 6 to 14 carbon atoms such as phenyl, naphthyl, tetrahydronapthyl, indanyl, and biphenyl.

The term “arylalkyl” refers to an aryl group as defined above directly bonded to an alkyl group as defined above e.g., —CH₂C₆H₅ and —C₂H₅C₆H₅.

The term “heterocyclic ring” refers to a stable 3- to 15-membered ring radical which consists of carbon atoms and from one to five heteroatoms selected from nitrogen, phosphorus, oxygen and sulfur. For purposes of this invention, the heterocyclic ring radical may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heterocyclic or heteroaryl). Examples of such heterocyclic ring radicals include, but are not limited to, azetidinyl, acridinyl, benzodioxolyl, benzodioxanyl, benzofumyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, quinoxalinyl, quinolinyl, isoquinolinyl, tetrazoyl, imidazolyl, tetrahydroisouinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, furyl, tetrahydrofurtyl, tetrahydropyranyl, thienyl, benzothienyl, thiamorpholinyl, thiamorpholinyl sulfoxide, thiamorpholinyl sulfone, dioxaphospholanyl, oxadiazolyl, chromanyl, and isochromanyl. The heterocyclic ring radical may be attached to the main structure at any heteroatom or carbon atom that results in the creation of a stable structure.

The term “heteroaryl” refers to an aromatic heterocyclic ring radical. The heteroaryl ring radical may be attached to the main structure at any heteroatom or carbon atom that results in the creation of a stable structure.

The term “heteroarylalkyl” refers to a heteroaryl ring radical directly bonded to an alkyl group. The heteroarylalkyl radical may be attached to the main structure at any carbon atom from the alkyl group that results in the creation of a stable structure.

The term “heterocyclyl” refers to a heterocyclic ring radical as defined above. The heterocyclyl ring radical may be attached to the main structure at any heteroatom or carbon atom that results in the creation of a stable structure.

The term “heterocyclylalkyl” refers to a heterocyclic ring radical directly bonded to an alkyl group. The heterocyclylalkyl radical may be attached to the main structure at any carbon atom in the alkyl group that results in the creation of a stable structure.

The term “alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, and 1,1-dimethylethyl (t-butyl).

The term “alkenyl” refers to an aliphatic hydrocarbon group containing a carbon-carbon double bond and which may be a straight or branched chain having 2 to about 10 carbon atoms, e.g., ethenyl, 1-propenyl, 2-propenyl (allyl), iso-propenyl, 2-methyl-1-propenyl, 1-butenyl, and 2-butenyl.

The term “alkynyl” refers to a straight or branched chain hydrocarbyl radical having at least one carbon-carbon triple bond, and having 2 to about 12 carbon atoms (with radicals having 2 to about 10 carbon atoms being preferred), e.g., ethynyl, propynyl, and butynyl.

The term “alkoxy” denotes an alkyl group attached via an oxygen linkage to the rest of the molecule. Representative examples of such groups are —OCH₃ and —OC₂H₅.

The term “cycloalkyl” denotes a non-aromatic mono or multicyclic ring system of 3 to about 12 carbon atoms, such as cyclopropyl, cyclobutyl, cyclopentyl, and cyclohexyl. Examples of multicyclic cycloalkyl groups include, but are not limited to, perhydronapththyl, adamantyl and norbornyl groups, bridged cyclic groups or sprirobicyclic groups, e.g., sprio (4,4) non-2-yl.

The term “cycloalkylalkyl” refers to a cyclic ring-containing radical, having 3 to about 8 carbon atoms, directly attached to an alkyl group. The cycloalkylalkyl group may be attached to the main structure at any carbon atom in the alkyl group that results in the creation of a stable structure. Non-limiting examples of such groups include cyclopropylmethyl, cyclobutylethyl, and cyclopentylethyl.

The term “cycloalkylaryl” refers to a cyclic ring-containing radical, having 3 to about 8 carbon atoms, directly attached to an aryl group. Non-limiting examples or such groups include phenylcyclopropyl, phenylcylobutyl and phenylcyclopentyl. The term “cycloalkenyl” refers to a cyclic ring-containing radical having 3 to about 8 carbon atoms with at least one carbon-carbon double bond, such as cyclopropenyl, cyclobutenyl, and cyclopentenyl.

Unless otherwise specified, the term “substituted” as used herein refers to substitution with any one or any combination of the following substituents: hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted or unsubstituted heterocyclylalkyl ring, substituted or unsubstituted heteroarylalkyl, substituted or unsubstituted heterocyclic ring, substituted or unsubstituted guanidine, —COORₓ, —C(O)Rₓ, —C(S)Rₓ, —C(O)NRₓR_{y}, —C(O)ONRₓR_{y}, —NRₓCONR_{y}R_{z}, —N(Rₓ)SOR_{y}, —N(Rₓ)SO₂R_{y}, —(═N—N(Rₓ)R_{y}), —NRₓC(O)OR_{y}, —NRₓR_{y}, —NRₓC(O)R_{y}, —NRₓC(S)R_{y}, —NRₓC(S)NR_{y}R_{z}, —SONRₓR_{y}, —SO₂NRₓR_{y}, —ORₓ, —ORₓC(O)NR_{y}R_{z}, —ORₓC(O)OR_{y}, —OC(O)Rₓ, —OC(O)NRₓR_{y}, —RₓNR_{y}C(O)R_{z}, —RₓOR_{y}, —RₓC(O)OR_{y}, —RₓC(O)NR_{y}R_{z}, —RₓC(O)R_{y}, —RₓOC(O)R_{y}, —SRₓ, —SORₓ, —SO₂Rₓ, and —ONO₂, wherein Rₓ, R_{y}, and R_{z} are independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted heterocyclylalkyl ring, substituted or unsubstituted heteroarylalkyl, or substituted or unsubstituted heterocyclic ring. According to one embodiment, the substituents in the aforementioned “substituted” groups cannot be further substituted. For example, when the substituent on “substituted alkyl” is “substituted aryl” the substituent on “substituted aryl” cannot be “substituted alkenyl”.

The term “protecting group” or “PG” refers to a substituent that is employed to block or protect a particular functionality. Other functional groups on the compound may remain reactive. For example, an “amino-protecting group” is a substituent attached to an amino group that blocks or protects the amino functionality in the compound. Suitable amino-protecting groups include, but are not limited to, acetyl, trifluoroacetyl, t-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable hydroxy-protecting groups include, but are not limited to, acetyl and silyl. A “carboxy-protecting group” refers to a substituent of the carboxy group that blocks or protects the carboxy functionality. Suitable carboxy-protecting groups include, but are not limited to, —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, and nitroethyl. For a general description of protecting groups and their use, see T. W. Greene, Protective Groups in Organic Synthesis, John Wiley & Sons, New York, 1991.

Of the various cannabinoid receptor agonist compounds disclosed in the PCT Application Publication No. WO 2006/129178, those of particular interest, in the context of present invention, include the ones which are selective CB2 receptor agonists. One such selective CB2 receptor agonist is N5-(tert-butyl)-3-(2,4-difluorophenyl)-3,4-diazatricylco[5.2.1.0^{2,6}]deca-2(6),4-diene-5-carboxamide or a pharmaceutically acceptable salt thereof. Another such selective CB2 receptor agonist is (1R,7S)—N-5-(tert-butyl)-3-(2,4-difluorophenyl)-3,4-diazatricylco[5.2.1.0^{2,6}]deca-2(6),4-diene-5-carboxamide (the compound “GRC-CPD-A”) including its pharmaceutically acceptable salts.

The term “cannabinoid receptor” refers to any one of the known or heretofore unknown subtypes of the class or cannabinoid receptors, including CB1 and/or CB2 receptors, that may be bound by a cannabinoid modulator compound of the present invention.

The term “modulator” further refers to a compound of the invention CB (e.g., CB1 and/or CB2) receptor agonist, partial agonist, antagonist or inverse-agonist.

The term “treating” or “treatment” of non-immediate type allergic disease or symptoms thereof includes: (1) preventing or delaying the appearance of clinical symptoms of the disease developing in a subject that may be afflicted with or predisposed to the disease but does not yet experience or display clinical or subclinical symptoms or the disease; (2) inhibiting arresting or reducing the development of the disease or at least one clinical or subclinical symptom thereof; or (3) relieving the disease, i.e., causing regression of the state, disorder or condition or at least one of its clinical or subclinical symptoms. The benefit to a subject to be treated is either statistically significant or at least perceptible to the subject or to the physician.

The term “subject” includes mammals (especially humans) and other animals, such as domestic animals (e.g., household pets including cats and dogs) and non-domestic animals (such as wildlife).

A “therapeutically effective amount” means the amount of a compound that, when administered to a subject for treating a state, disorder or condition, is sufficient to effect such treatment. The “therapeutically effective amount” will vary depending on the compound, the disease and its severity and the age, weight, physical condition and responsiveness of the subject to be treated.

Pharmaceutically acceptable salts forming part of this invention include salts derived from inorganic bases (such as Li, Na, K, Ca, Mg, Fe, Cu, Zn, and Mn), salts of organic bases (such as N,N′-diacetylethylenediamine, glucamine, triethylamine, choline, dicyclohexylamine, benzylamine, trialkylamine, and thiamine), salts of chiral bases (such as alkylphenylamine, glycinol, and phenyl glycinol), salts of natural amino acids (such as glycine, alanine, valine, leucine, isoleucine, norleucine, tyrosine, cystine, cysteine, methionine, proline, hydroxy proline, histidine, ornithine, lysine, arginine, and serine), salts of non-natural amino acids (such as D-isomers or substituted amino acids), salts of guanidine, salts of substituted guanidine (wherein the substituents are selected from nitro, amino, alkyl, alkenyl, or alkynyl), ammonium salts, substituted ammonium salts, and aluminum salts. Other pharmaceutically acceptable salts include acid addition salts (where appropriate) such as sulphates, nitrates, phosphates, perchlorates, borates, hydrohalides, acetates (such as trifluoroacetate), tartrates, maleates, citrates, fumarates, succinates, palmoates, methanesulphonates, benzoates, salicylates, benzenesulfonates, ascorbates, glycerophosphates, and ketoglutarates. Yet other pharmaceutically acceptable salts include, but are not limited to, quaternary ammonium salts of the compounds of invention with alkyl halides or alkyl sulphates (such as MeI or (Me)₂SO₄).

Pharmaceutically acceptable solvates includes hydrates and other solvents of crystallization (such as alcohols). The compounds of the present invention may form solvates with standard low molecular weight solvents by methods known in the art.

As used herein the term “allergic disease” refers to any disease associated with an allergic reaction. For example, allergic diseases include allergic diseases of skin and respiratory organs. Allergic diseases which can be treated by the present invention include, but are not limited to, anaphylaxis, digestive tract allergy, allergic gastritis, allergic dermatitis, atopic dermatitis, asthma, allergic asthma, atopic asthma, allergic bronchial pulmonary aspergillosis, pollenosis, allergic rhinitis, allergic conjunctivitis, allergic sarcoma angitis, chemical allergy and serum disease. According to a specific embodiment, the allergic disease is allergic asthma, allergic dermatitis or atopic dermatitis.

As used herein the term “non-immediate type allergic disease” refers to an allergic disease involving a late phase allergic reaction and/or delayed-type allergic reaction, which may or may not be accompanied by an immediate type allergic reaction.

The term “immediate type allergic reaction” refers to an allergic reaction in which symptoms are exhibited within two hours of exposure to an allergen, and typically within thirty minutes of exposure.

As used herein the term “late phase allergic reaction” refers to an allergic reaction in which symptoms are exhibited more than two hours to a few days alter exposure to an allergen.

As used herein the term “delayed type allergic reaction” refers to an allergic reaction in which symptoms are exhibited more than a few days, for example, about 2 days to about 10 days, after exposure to an allergen.

As used herein the term “allergic dermatitis” refers to dermatitis associated with an allergic reaction and includes, for example, atopic dermatitis.

As used herein the term “allergic asthma” refers to asthma symptoms triggered by an allergic reaction and includes, for example, mixed type asthma and atopic asthma.

As used herein the term “selective CB2 receptor agonist” refers to a CB2 receptor agonist which selectively binds the CB2 receptor over the CB1 receptor. The binding affinity of a CB2 receptor agonist for the hCB1 and hCB2 receptors can be measured by protocols II and IV of International Publication No. WO 2006/129178 (17 (pages 193-5). These protocols measure the percent displacement of a radioligand which binds hCB1 or hCB2 receptor by a test compound. Preferably, the ratio of the percent displacements for hCB1 to hCB2 receptor as measured by protocols II and IV is at least 1:3, more preferably at least 1:5, and even more preferably at least 1:8.

An aspect of the present invention provides a method of treating a non-immediate type allergic disease in a mammal by administering to the mammal a selective CB2 receptor agonist, such as N5-(tert-butyl)-3-(2,4-difluorophenyl)-3,4-diazatricylco[5.2.1.0^{2,6}]deca-2(6),4-diene-5-carboxamide, including its pharmaceutically acceptable salts, (1R,7S)—N5-(tert-butyl)-3-(2,4-difluorophenyl)-3,4-diazatricylco[5.2.1.0^{2,6}]deca-2(6),4-diene-5-carboxamide or a pharmaceutically acceptable prodrug, pharmaceutically acceptable N-oxide, or pharmaceutically acceptable salt thereof.

Another aspect of the present invention provides a method of using a selective CB2 receptor agonist, such as N5-(tert-butyl)-3-(2,4-difluorophenyl)-3,4-diazatricylco[5.2.1.0^{2,6}]deca-2(6),4-diene-5-carboxamide, including its pharmaceutically acceptable salts, (1R,7S)—N5-(tert-butyl)-3-(2,4-difluorophenyl)3,4-diazatricylco[5.2.1.0^{2,6}]deca-2(6),4-diene-5-carboxamide or a pharmaceutically acceptable prodrug, pharmaceutically acceptable N-oxide, or pharmaceutically acceptable salt thereof for the treatment of a non-immediate type allergic disease in a mammal. Particularly, the CB2 receptor agonist is administered to the mammal by the oral, parenteral, or topical route.

The compound of the present invention can be administered directly to the subject to achieve the therapeutic goal of the present invention. Alternatively, the compound can be administered in the form of a pharmaceutical composition, which comprises one or more compounds described herein, and one or more pharmaceutically acceptable excipients, carriers, diluents or mixtures thereof.

A further aspect of the present invention provides a pharmaceutical formulation comprising a selective CB2 receptor agonist, such as N5-(tert-butyl)-3-2,4-difluorophenyl)-3,4-diazatricylco[5.2.1.0^{2,6}]deca-2(6),4-diene-5-carboxamide, (1R,7S)—N5-(tert-butyl)-3-(2,4-difluorophenyl)-3,4-diazatricylco[5.2.1.0^{2,6}]deca-2(6),4-diene-5-carboxamide or a pharmaceutically acceptable prodrug, pharmaceutically acceptable N-oxide, or pharmaceutically acceptable salt thereof optionally together with one or more pharmaceutically acceptable excipients, carriers, diluents or mixtures thereof, for treatment of a non-immediate type allergic disease in a mammal

Yet another aspect of the present invention provides use of a selective CB2 receptor agonist, such as N5-(tert-butyl)-3-(2,4-difluorophenyl)-3,4-diazatricylco[5.2.1.0^{2,6}]deca-2(6),4-diene-5-carboxamide, (1R,7S)—N5-(tert-butyl)-3-(2,4-difluorophenyl)-3,4-diazatricylco[5.2.1.0^{2,6}]deca-2(6),4-diene-5-carboxamide or a pharmaceutically acceptable prodrug, pharmaceutically acceptable N-oxide, or pharmaceutically acceptable salt thereof for preparing a pharmaceutical formulation for treatment of a non-immediate type allergic disease in a mammal.

The pharmaceutical composition described herein is administered to a subject by any of the following routes, for example, oral, parenteral or topical. Parenteral routes comprise intravenous, intraarterial, intraperitoneal, intrathecal, intraventricular, intraurethral, intrasternal, intracranial, intramascular, subcutaneous, intranasal, inhalation, ophthalmic, rectal or vaginal routes. Particularly, the route of administration is topical including transdermal, and/or oral including buccal. The compounds described herein may be administered alone but will generally be administered as an admixture with a suitable pharmaceutically acceptable carrier such as non-toxic, inert solid, semi solid or liquid filter, diluent, encapsulating material or formulation auxiliary of any type.

The pharmaceutical compositions are in conventional forms, for example, solid form preparation (e.g. capsules, tablets, pills, powder, granules or suppositories), liquid form preparation (e.g., aerosols, emulsions, solutions, suspensions, syrups or elixirs), injectible preparation (e.g., sterile injection or aqueous or oleaginous suspensions), topical or transdermal preparation (e.g., ointments, pastes, creams, lotions, gel, powder, solutions, spray, inhalants or patches). More particularly, the composition of the present invention is in the form of solid preparation including tablet, capsule, granules, beads or pellets, powder, dry syrup for suspension, and the like, and their combinations; or topical preparation including gel, lotion, aerosols, emulsions, solutions, suspensions, ointments, pastes, creams, spray, patches and their combinations.

The pharmaceutical compositions of the present invention are prepared by conventional techniques known to those skilled in the art including, for example, the methods described in Remington: The Science and Practice of Pharmacy, 20^{th} Ed., 2003 (Lippincott Williams & Wilkins).

For solid form preparation, the active ingredient is mixed with at least one pharmaceutically acceptable carrier or excipient such as sodium citrate, dicalcium phosphate and/or a filler or extender such as starch, lactose, sucrose, glucose, mannitol, or salicic acid; binders such as carboxymethyl cellulose, alginates gelatins, polyvinylpyrrolidinone, sucrose, acacia: disintegrating agents such as agar-agar, calcium carbonate, potato starch, alginic acid, certain silicates or sodium carbonate; absorption accelerators such as quaternary ammonium compounds; wetting agents such as cetyl alcohol, glycerol, monostearate: adsorbents such as kaolin; lubricants such as talc, calcium stearate, magnesium stearate, solid polyethylene glycol, sodium lauryl sulphate, or mixture thereof.

For liquid form preparation, the active compound is mixed with water or other solvent, solubilizing agents and emulsifiers such as ethyl alcohol, isopropyl alcohol, ethyl carbonate, ethyl acetate, benzyl alcohol, benzyl benzoate, propylene glycol, dimethylformamide, glycerol, fatty acid ester of sorbitan, oils such as cottonseed, groundnut, corn, germ, olive, castor or sesame oil, or mixture thereof. The oral compositions may also include adjuvants such as wetting agents, emulsifying agents, suspending agents, sweetening agents, or flavouring agents, or perfuming agents.

Injectable preparation may be formulated according to the methods known in the art, for example, using suitable dispersing or wetting and suspending agents. Water, Ringer's solution and isotonic sodium chloride may be employed as acceptable vehicles. For topical or transdermal preparation, the active compound is admixed under sterile condition with a pharmaceutically acceptable carrier and any needed preservatives or buffer as may be required.

The formulation as described herein is formulated so as to provide quick, sustained or delayed release of the active compound after administering to a subject in need thereof by using procedures well known to those skilled in the art of formulary. Thus, in addition to the common dosage forms set out above, the active ingredient in therapeutic amount may be administered by controlled release means and/or delivery devices to provide the rate controlled release of any one or more of the active compounds to optimize the desired therapeutic effect. Suitable dosage forms for sustained release include layered tablets containing layers of varying disintegration rates or controlled release polymeric matrices impregnated with the active compound and shaped in tablet form or capsules containing such impregnated or encapsulated porous polymetric matrices.

The magnitude of a prophylactic or therapeutic dose of one or more compounds described herein in the management of disease or disorder will vary with the severity of the condition to be treated and the route of administration. The dose and the dose frequency will also vary according to the age, body weight, and response of the individual. The dosage ranges can easily be determined by those skilled in the art.

Suitable doses of the compounds for use in treating non-immediate type allergic disease or symptoms thereof can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms, suitable pharmaceutical excipients, diluents or carriers can also be well used and adjusted by those skilled in the art. All changes and modifications are envisioned within the scope of the present invention.

The pharmaceutical composition may contain a compound of formula (I) as an active ingredient upto 90 percent to the total weight of the formulation. More particularly, the pharmaceutical composition may contain a compound of formula (I) as an active ingredient upto 70 percent to the total weight of the formulation. Specifically, the pharmaceutical composition may contain a compound of formula (I) as an active ingredient upto 50 percent to the total weight of the formulation. When the composition contains two compounds of formula (I) as active ingredients, they may be in the ratios from about 1:10 to 10:1. Further, the ratios may be 1:1, 2:1, 1:2, 1:5 or even 5:1.

The present invention is described in detail below with reference to Example, but is not construed as being limited thereto.

### EXAMPLE

### Effect of GRC-CPD-A on the Inflammatory Reaction in a Model of toluene-2,4-diisocyanate (TDI) Induced Dermatitis in Female BALB/c Mice

### Materials and Methods

Toluene-2,4-diisocyanate (TDI), O-phenyl diethylamine and urethane were purchased from Sigma Chemicals, USA. Leishman's stain and acetone were purchased from Qualigens, India and DMSO was purchased from Merck, India. GRC-CPD-A (batch VIII-R) was prepared in-house by the discovery chemistry group.

### Animals

Healthy female BALB/c mice weighing between 15-20 g were obtained from ACTREC, Kharghar.

### Animal Breeding and Maintenance

The mice were housed in the Glenmark Animal Facility. The facility has been registered for ‘Research & Breeder of Animals’ by the Committee for the Purpose of Control and Supervision of Experiments on Animals (CPCSEA), Registration number 231/2000/CPCSEA.

Animals were group housed in polypropylene cages. They were housed at a constant temperature of 23±1° C. with a light/dark cycle of 12/12 hours. They were given a standard rodent pellet diet and water ad libitum. Standard hygiene procedures and animal welfare guidelines as prescribed by the Institutional Animal Ethics Committee (IAEC) were followed.

### Active Sensitization

The sensitization protocol was as per W. Baumer et al., J Pharm Pharmacol., 55(8):1107-14 (2003). The abdominal region was shaved and depilated using Veet. Subsequently, the horny layers were stripped 10 times with adhesive tape. For active sensitization, 100 μl of 5% TDI was applied to the stripped skin for 3 consecutive days.

Prescreening was performed 21 days later. In brief, the sensitized mice were challenged with an application of 10 μl of 0.5% TDI on both the inner and outer surface of the left ear, the right ear served as control in each mouse. Ear thickness was measured with a cutimeter (Model7309, Mitutoyo Japan) prior to challenge and 24 hours after challenge. Animals that had a mean swelling difference of 20% or greater at 24 hours when compared to basal value were considered to be sensitized and were distributed to various treatment groups. Animals were rested for 7 days after prescreening, to allow for the ear thickness to return to normal.

### Animal Groupings

Five groups (A-E) were studied. Details of the groups are shown in Table 1.

TABLE 1

| Group | Group Code* | Animals | Treatment |
|---|---|---|---|
| A | V/TDI/Acetone | 5 | Vehicle |
| B | V/TDI/TDI | 5 | Vehicle |
| C | GRC-CPD-A/TDI/TDI | 8 | 0.3% GRC-CPD-A, |
| | | | topical |
| D | GRC-CPD-A/TDI/TDI | 8 | 3% GRC-CPD-A, |
| | | | topical |
| E | GRC-CPD-A/TDI/TDI | 8 | 10% GRC-CPD-A, |
| | | | topical |

*A: Vehicle treated/: Toluene-2,4-diisocyanate sensitized/acetone challenged (V/TDI/Acetone); B: Vehicle treated/Toluene-2,4-diisoscyanate sensitized/Toluene-2,4-diisocyanate challenged (V/TDI/TDI); C: 0.3% GRC-CPD-A/Toluene-2,4-diisocyanate sensitized/Toluene-2,4-diisocyanate challenged; D: 3% GRC-CPD-A/Toluene-2,4-diisocyanate sensitized/Toluene-2,4-diisocyanate challenged; and E: 10% GRC-CPD-A/Toluene-2,4-diisocyanate sensitized/Toluene-2,4-diisocyanate challenged

### Compound Administration & TDI Challenge

On day 28, test compounds. GRC-CPD-A (0.3, 3 and 10%) were prepared using DMSO and acetone in the ratio of 1:9 as solvents. 20 μl (10 μl on each side of the ear) of the formulation was applied to the left ear of each mouse as per the study design 6 hours prior to challenge with TDI. 20 μl of TDI was applied to both inner and outer surface of the left ear of each mouse. The right ear served as control and received an application of 10 μl of acetone on each side. 24 hours post TDI challenge ear thickness was determined and ears were collected for histology and eosinophil peroxidase (EPO) determination. Ear sections were processed for histology and stained with hematoxylin-eosin (HE) and lesions were scored as follows: 0=no cell infiltration or edema, 1-4-granulocyte infiltration/oedema depending on severity. A combined congo-red-toluidine blue staining was also carried out to identify eosinophils which were then quantified per 12 high power fields (hpf). EPO levels in ear sections were determined by standard calorimetric assay using O-phenyl diethylamine as substrate.

### Data Analysis:

All data were expressed as mean±SEM. All groups were subjected to one-way ANOVA and means were compared using Dunnett's post hoc test using GraphPad prism software.

### Results:

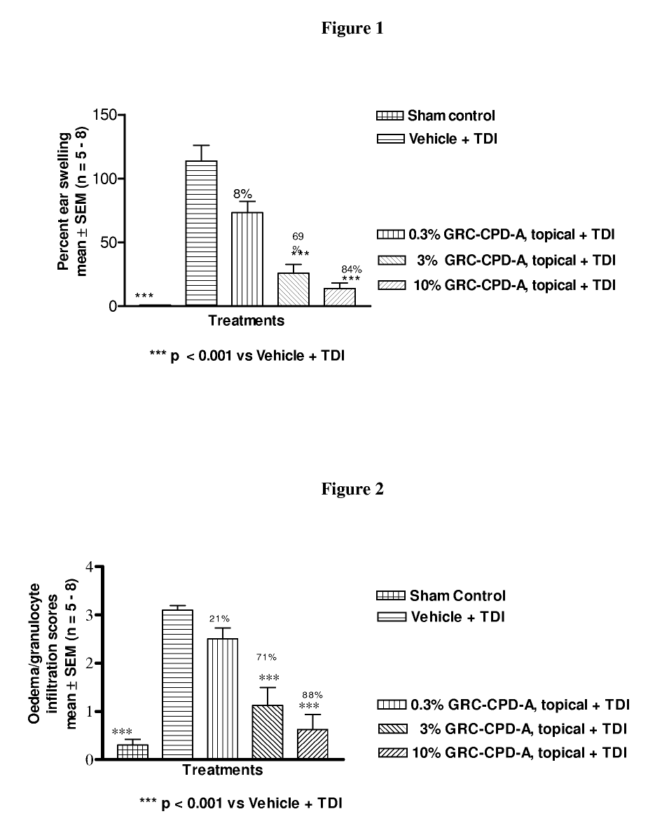

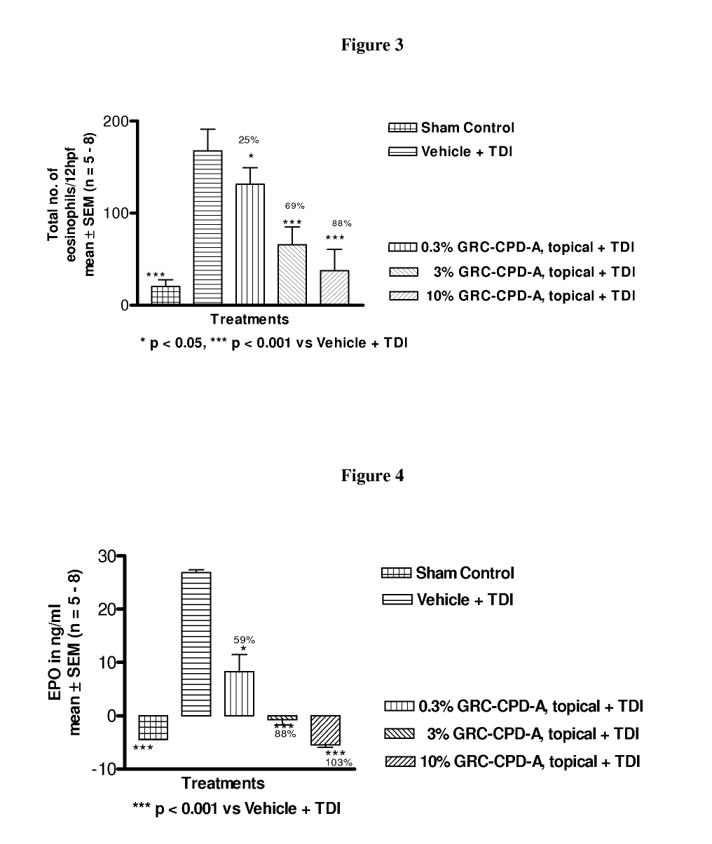

Topical application of 10% GRC-CPD-A produced significant (p<0.001) dose-dependent inhibition of ear swelling, improved histological scores, inhibited eosinophil infiltration and inhibited EPO levels in the ear (FIGS. 1, 2, 3 & 4). Standard reference compounds tacrolimus and hydrocortisone also produced significant inhibition of various parameters.

### CONCLUSION

This study shows that GRC-CPD-A a selective agonist for CB2 receptor effectively inhibits ear swelling (oedema and eosinophil infiltration) and EPO levels in a dose dependent manner and acts as a potent anti-inflammatory agent in the TDI induced atopic dermatitis model in BALB/c mice.

1. A method of treating a non-immediate type allergic disease in a mammal comprising administering to the mammal a therapeutically effective amount of a selective CB2 receptor agonist, wherein the selective CB2 receptor agonist is N5-(tert-Butyl)-3-(2,4-difluorophenyl)-3,4-doazatricylco[5.2.1.0^{2,6}]deca-2(6),4-diene-5-carboxamide or a pharmaceutically salt thereof.

2. The method of claim 1, wherein the selective CB2 receptor agonist is (1R,7S)—N5-(tert-butyl)-3-(2,4-difluorophenyl)-3,4-diazatricylco[5.2.1.0^{2,6}]deca-2(6),4-diene-5-carboxamide or a pharmaceutically acceptable salt thereof.

3. A method according to claim 1, wherein the non-immediate type allergic disease is allergic asthma, allergic dermatitis, or atopic dermatitis.

4. A method according to claim 1, wherein the mammal is a human.

5. A method according to claim 1, wherein the selective CB2 receptor agonist is administered orally, topically or parenterally.

6. A method according to claim 2, wherein the non-immediate type allergic disease is allergic asthma, allergic dermatitis, or atopic dermatitis.

7. A method according to claim 2, wherein the mammal is a human.

8. A method according to claim 2, wherein the selective CB2 receptor agonist is administered orally, topically or parenterally.

9. A pharmaceutical composition comprising a therapeutically effective amount of N5-(tert-butyl)-3-(2,4-difluorophenyl)-3,4-diazatricylco[5.2.1.0^{2,6}]deca-2(6), 4-diene-5-carboxamide or a pharmaceutically acceptable salt thereof, for treating a non-immediate type allergic disease in a mammal.

10. A pharmaceutical composition according to claim 9, wherein the non-immediate type allergic disease is allergic asthma, allergic dermatitis, or atopic dermatitis.

11. A pharmaceutical composition according to claim 9, wherein the mammal is a human.

12. A pharmaceutical composition according to claim 9, wherein the composition is administered orally, topically or parenterally.

13. A pharmaceutical composition comprising a therapeutically effective amount of (1R,7S)—N-5-(tert-butyl)-3-(2,4-difluorophenyl)-3,4-diazatricylco[5.2.1.0^{2,6}]deca-2(6),4-diene-5-carboxamide or a pharmaceutically acceptable salt thereof, for treating a non-immediate type allergic disease in a mammal.

14. A pharmaceutical composition according to claim 13, wherein the non-immediate type allergic disease is allergic asthma, allergic dermatitis, or atopic dermatitis.

15. A pharmaceutical composition according to claim 13, wherein the mammal is a human.

16. A pharmaceutical composition according to claim 13, wherein the composition is administered orally, topically or parenterally.
